(19) United States
(12) Patent Application Publication
(10) Pub. No.: US 2015/0289962 A1
(43) Pub. Date: Oct. 15, 2015

(54) Livestock Treatment Method and Apparatus

(71) Applicant: R & M BUTTERWORTH PTY LTD., Victoria (AU)

(72) Inventor: Richard John Butterworth, Victoria (AU)

(73) Assignee: R & M BUTTERWORTH PTY LTD., Victoria (AU)

(21) Appl. No.: 14/251,604

(22) Filed: Apr. 13, 2014

(51) Int. Cl. A61D 7/00; A62B 31/00; A61M 16/10; A61G 10/02

(52) U.S. Cl. CPC A61D 7/00; A61G 10/023; A62B 31/00; A61M 16/10; A61M 2202/066; A61M 2250/00

(57) ABSTRACT

A method of contacting the cell surfaces of livestock which are in contact with the atmosphere, with firmly divided mineral salts in airborne suspension, by confining the animal in a chamber and exposing the animal to an airstream containing the suspension. The method makes use of a chamber with a circulation fan. The chamber may take single animals or groups. Usually the chamber is a static installation but may be adopted to trailer form for treating animals during the journey or transporting the facilities in a vehicle for treating zoo animals example or bees.

### TECHNICAL FIELD

This invention concerns a treatment method for animals and apparatus for performing the method.

The method is intended for horses and greyhounds but may be beneficial to livestock generally and cattle in particular. A large proportion of the body weight of a racehorse and greyhound is skeletal muscle. They have large lung capacity to match because the combination permits them to run at speed. To maximise and maintain speed, the respiratory system must work perfectly and performance depends on the airways maximising the flow of air to the lungs. Any constriction leads to panting and reduction in performance.

### BACKGROUND

The extent and frequency of impairment is only evident to trainers who are in constant contact with the horses and dogs in their care. When skin ailments are active they are visible as rashes, surface ulcers, wounds, bruises, swelling and the like and topical or systemic treatment can be planned. When respiratory problems are spoiling the animals performance or recovery from training the cause is often discernable. Heavy panting may indicate blockage of airways with mucus or sputum. While infections by bacteria may be treatable with antibiotics, these frequently disturb the animal through side effects. If the infection is viral the position is less treatable because there are relatively few anti-virals. So there is a need for a treatment which is rapid in effect and simple to administer.

A veterinary inspection is a usual step and the introduction of a diagnostic camera helps to show to what extent the airways are not performing as they should. This sort of inspection may be done between training sessions. Once the airways are shown to be the source of slow speed rather than body pain or systemic infection then localised treatment can be planned.

Trainers like treatments which are rapid in effect so that time lost in training is minimised. This is particularly so when a horse is recovering from travel fatigue. This condition is common and can subtract severely from a training program. This shows as general debility and may last for weeks.

### SUMMARY OF INVENTION

The apparatus aspect of the invention provides a chamber for temporary accommodation of one or more animals to be treated with means to cause an airstream in the chamber and dispensing means for adding finely divided mineral salts which form a suspension when dispensed into the airstream.

The dispensing means may include a comminuter for reducing the crystalline or amorphous salts to a particle size suitable for air suspension, namely 5-15Φ.

The airstream may be created by a fan such as an axial flow fan.

The chamber may have surfaces which resist deposition of the salt particles.

The chamber may have a desiccator which can be covered and uncovered in order to remove excess water vapour from the air.

The chamber may have floor cleaning apparatus for example a mobile manure scoop.

The method aspect of the invention provides a method of contacting the cell surfaces of livestock which are in contact with the atmosphere with finely divided mineral salts in airborne suspension, including the steps of confining the animal in a chamber and exposing the animal to an airstream which contains the suspension, whereby the salts are inspired and optionally repeating the exposure.

The method further includes a dispersing finely ground mineral salts in an airstream created in a chamber containing an animal to be treated and keeping the animal in the chamber for a period sufficient for the salts to be consumed by inspiration of the animal or deposited on the animals skin.

A mixture of salts are preferable. The dose may vary according to the severity of the condition 5 g, 10 g, 15 g and 20 g/m³ being the preferred range. The frequency may also vary according to the severity and may be daily or 1-6 sessions per week. The sessions may last 15-45 minutes.

Periodically the air in the chamber may be exposed to a desiccator in order to reduce the humidity caused by the expiration and perspiration of the animals.

The walls, floors and ceiling tend to become coated with the salts particles and those which contain water of crystallization such as magnesium salts cause moist surfaces to persist. Some salts are deliquescent and these produce a water film on the floor. It is useful to dust the chamber floor with common salt, preferably sea salt and a desiccating agent such as calcium chloride.

The salt for airborne suspension may be phosphates, sulphates, chlorides and fluorides of sodium, potassium, calcium, magnesium and iron.

### Advantageous Effects of Invention

1. Respiratory problems clear up and a return to training occurs sooner. Bleeding and mucus production in the airways diminishes. Attacks of pharyngitis moderate.

2. Travel sickness is dispelled.

3. Skin complaints are alleviated.

### BRIEF DESCRIPTION OF DRAWINGS

The embodiment of the invention is now described with reference to the accompanying drawings in which:

FIG. 1 is a plan of a therapy chamber for three horses with adjoining staff and record space.

FIG. 2 is a side view of the chamber in FIG. 1.

FIG. 3 is the salt dispensing apparatus.

FIG. 4 is a plan of a chamber for a larger number of horses.

### DESCRIPTION OF EMBODIMENTS

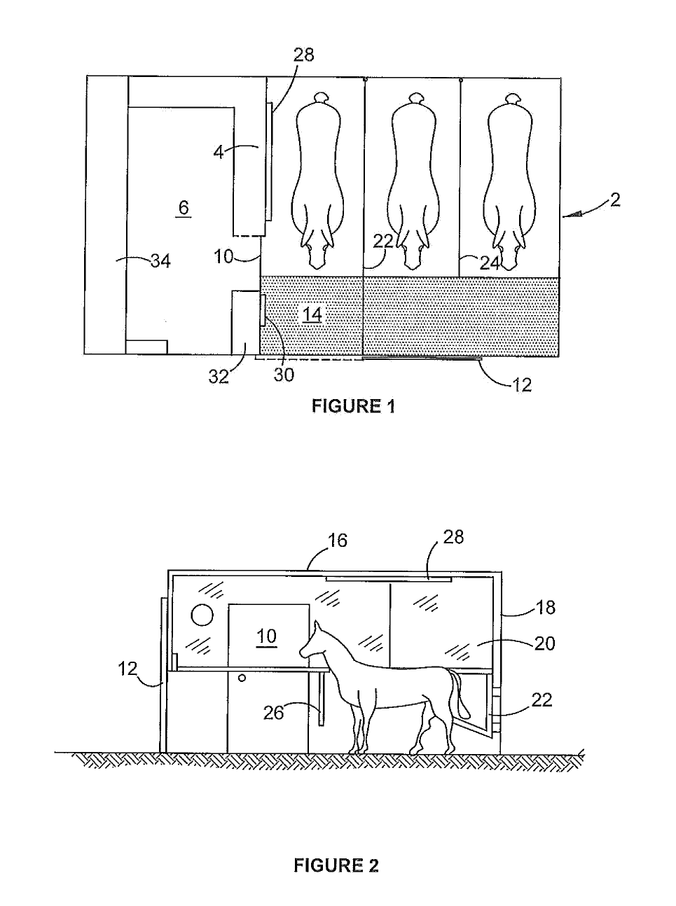

Referring to FIGS. 1 and 2, the treatment chamber 2 is a room 5 m×4.5 m with a partition wall 4 which divides the staffroom 6 from the animal area 8. Door 10 allows access from the staffroom to the animal area. Door 12 acts as entrance and exit to the animals. The floor is concrete and a strip lm wide 14 is covered with chemical desiccant to a depth of 20-25 mm. This is a mixture of vermiculite, perlite and calcium chloride.

The ceiling 16 and upper walls 18 are covered with panels 20 which are coated with desiccant but which after initial use become coated with a second layer of treatment salts.

Gate 22 pivots left and right and fastens to the frame of door 10. Shorter gate 24 divides the animal space into a second and third stalls for accommodating two more animals. Collar ties 26 hang from the gates to restrain lively animals. A fluorescent light 28 illuminates the chamber.

Partition wall 4 has an aperture 30 through which air is blown by the salt dispenser 32 mounted on the wall of the staffroom 6. Desk space and record keeping containers 34 occupy one wall of the staffroom.

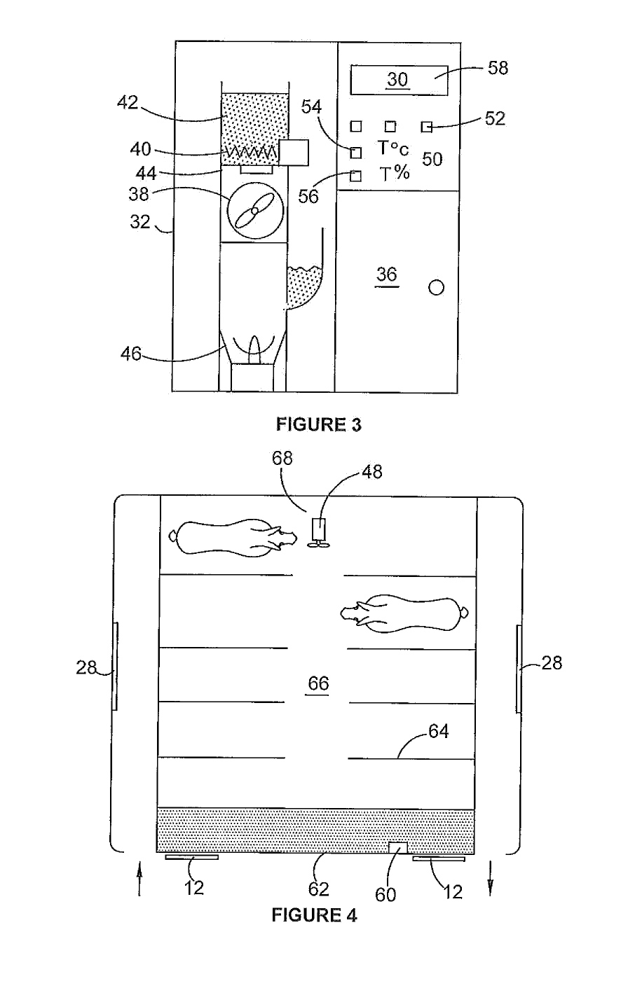

Referring now to FIG. 3, the wall mounted salt dispenser 32 has a storage compartment 36 beside the axial flow fan 38. Above the fan is a grinder 40 having an electrically driven screw which is fed by a reservoir 42 of about 1 kg capacity. The screw reduces any lumps to a fluent crystalline mix which falls into a collector drawer 44 beneath the grinder.

The contents of the drawer are transferred to a measuring cup (not shown) and then tipped into a comminuter 46 which has electrically driven cutting blades. A restrictor causes the crystals to fall by gravity into the blades' path in a steady stream. The blades propel the powder produced by the comminuter upwardly into the airstream generated by an axial flow fan 38.

The fan is of 500 l/min capacity. The airstream blows the powdered salt into the animal area 8 where it circulates returning through the door 10 which is left ajar.

In practice, a batch of 60 g is prepared and increments of this powder are allowed to rise intermittently into the air flow over a period of minutes once the breathing rate of the horses has fallen to normal. The treatment continues for 15, 30 or 45 minutes and is repeated daily.

The electronic timer 50 allows the operator to select the treatment period. An air conditioner circuit (not shown) is controlled by a thermostat to adjust the chamber temperature. Humidity is kept low by the removal of desiccant. Buttons 52 select 15, 30 or 45 minute exposure. Buttons 54, 56 cause temperature and humidity to be shown on screen 58.

Three treatments on three days suffice to dispel travel sickness. A useful optional adjunct to the treatment is a negative-ion generator 60 on the opposite wall 62.

Referring now to FIG. 4, for large scale use, such as riding schools and racehorse training centres, a larger chamber is more suitable. The chamber shown is 7 m×7 m divided by rails 64 into spaces for individual horses.

The rails are in two rows divided by a central gap 66. At one end of the gap there is an axial flow fan 48 of 1000 l/min capacity which directs an airstream toward the opposite wall 58 through gap 66. The air circulates and reaches the space 68 behind the fan.

In a non-illustrated variant, the chamber is a trailer-mounted box like a horse box so that it may be towed by a vehicle to reach the animal, for example a zoo animal. In a further adaptation the mobile chamber is capable of treating one or more animals in the course of the journey. The chamber may alternatively form the body of a truck.

Treatment of camels with conventional medicines may affect the liver and other organs so salt therapy delivered in a quiet chamber like ours offers no harm and the ability to treat the whole animal.

The trailer may also be taken to apiaries so that hives may be stacked inside the box.

It is to be understood that the word “comprising” as used throughout the specification is to be interpreted in its inclusive form, ie. use of the word “comprising” does not exclude the addition of other elements.

It is to be understood that various modifications of and/or additions to the invention can be made without departing from the basic nature of the invention. These modifications and/or additions are therefore considered to fall within the scope of the invention.

I claim:

1. A method of contacting the cell surfaces of livestock which are in contact with the atmosphere with finely divided mineral salts in airborne suspension including the steps of confining the animal in a chamber and exposing the animal to an airstream which contains the suspension and optionally repeating the exposure.

2. A method as claimed in claim 1 plus the step of dispersing finely ground mineral salts in an airstream created in a chamber containing an animal to be treated and keeping the animal in the chamber for a period sufficient for the salts to be consumed through inspiration by the animal or deposition on the animals.

3. A method as claimed in claim 2, wherein 5, 10, 15, 20 g/m³ of the chamber volume are dispensed per treatment period.

4. A method as claimed in claim 3, wherein the treatment period may last 15-45 minutes.

5. A method as claimed in claim 2, wherein treatment is daily.

6. A method as claimed in claim 3, wherein the air in the chamber is exposed to a desiccation agent to reduce its humidity.

7. A method as claimed in claim 6, wherein the chamber floor is spread with a particulate drying agent.

8. A method as claimed in claim 7, wherein the drying agent is calcium chloride.

9. A method as claimed in claim 1, wherein the mineral salts are phosphates, sulphates, chlorides and fluorides of sodium, potassium, calcium, magnesium and iron.

10. A chamber for temporary accommodation of one or more animals to be treated with means to cause an airstream in the chamber and dispensing means for adding finely divided mineral salts which form a suspension when dispensed into the airstream.

11. A chamber as claimed in claim 10, wherein dispensing means includes a comminutor which reduces the crystalline or amorphous salts to a particle size suitable for air suspension.

12. A chamber as claimed in claim 11, wherein the particle size is 5-15Φ.

13. A chamber as claimed in claim 11, wherein the airstream is created by a fan.

14. A chamber as claimed in claims 10, wherein the chamber has surfaces which resist deposition of the mineral salt particles.

15. A chamber as claimed in claim 10, wherein the chamber ceiling and upper part of the walls are covered with removable panels.

16. A chamber as claimed in claim 10, wherein at least part of the chamber floor is impervious in order to retain desiccant.

17. A chamber as claimed in claims 10 when adapted as a trailer body or truck body.
